(19) United States
(12) Patent Application Publication
(10) Pub. No.: US 2022/0313604 A1
(43) Pub. Date: Oct. 6, 2022

(54) ANTISEPTIC AND/OR ANTI-INFLAMMATORY GEL FOR TREATMENT OF ORAL CAVITY DISEASES COMPRISING POLYVINYL ALCOHOL AND POLYACIDS

(71) Applicant: DTECH - SOCIETÀ A RESPONSABILITÀ LIMITATA, MAGLIE (LE) (IT)

(72) Inventor: Piero CHIARELLI, MAGLIE (LE) (IT)

(21) Appl. No.: 17/807,399

(22) Filed: Jun. 17, 2022

Related U.S. Application Data
(63) Continuation of application No. 16/982,949, filed on Sep. 21, 2020.

(30) Foreign Application Priority Data
Mar. 22, 2018 (IT) 102018000003872

(51) Int. Cl. A61K 9/06; A61P 1/02; A61K 6/20; A61K 6/69; A61K 8/04; A61K 8/22; A61K 8/36; A61K 8/368; A61K 8/43; A61K 8/73; A61K 8/81; A61K 9/00; A61K 31/155; A61K 31/192; A61K 31/616; A61K 47/32; A61K 47/36; A61Q 11/00

(52) U.S. Cl. CPC A61K 9/06; A61P 1/02; A61K 6/20; A61K 6/69; A61K 8/042; A61K 8/22; A61K 8/36; A61K 8/368; A61K 8/43; A61K 8/735; A61K 8/8129; A61K 8/8147; A61K 9/0063; A61K 31/155; A61K 31/192; A61K 31/616; A61K 47/32; A61K 47/36; A61Q 11/00

(57) ABSTRACT

Product for the therapeutic treatment for the prevention and cure of oral cavity diseases of teeth and dental implants including a mixture in a polyvinyl alcohol aqueous solution, of one or more polyacids or their salts, of one or more antiseptics and/or anti-inflammatories, capable of obtaining in situ a protective and adhesive barrier, capable of facilitating the bioavailability of the aforementioned medicinal products, antiseptics and anti-inflammatories.

### CROSS REFERENCE TO RELATED APPLICATIONS

This application is a Continuation of application Ser. No. 16/982,949, filed on Sep. 21, 2020, which is the National Phase under 35 U.S.C. § 371 of International Application No. PCT/IB2019/051880, filed on Mar. 8, 2019, which claims the benefit under 35 U.S.C. § 119(a) to patent application Ser. No. 102018000003872, filed in Italy on Mar. 22, 2018, all of which are hereby expressly incorporated by reference into the present application.

### TECHNICAL FIELD

The present invention patent application generally refers to the field of therapeutic treatments for the prevention and care of oral cavity diseases, teeth and dental implants and more in particular, the invention regards the prevention and care of alveolitis, and dental granuloma, dental cysts or infections after dental extraction or following a major dentistry intervention, infections among fixed or moveable dental prosthesis carriers, gingival recession, tartar or plaque build-up and halitosis.

The invention could apply to any industry, where such type of invention can be advantageously used, but it particularly generally refers to the stomatology industry.

### PRIOR ART

As known, oral cavity inflammations regarding the teeth, due to poor hygiene conditions, share the common trait of the progressiveness of the seriousness of the disease over time which, though treated with the current techniques, ends up creating progressive damage consisting in the mobilisation and loss of the tooth or implant.

Currently known in the prior art are numerous types of prevention and care preparations and relative uses in the stomatology industry.

The United States patent application n° US 200970148586 describes an injectable solution comprising collagen, chitosan, fibrinogen, alginate, calcium chloride, dental pulp stem cells and at least the association of two antibiotics.

The United States patent US 2009/0148486 describes the treatment of dental diseases through topical administration of metronidazole benzoate and chlorhexidine gluconate in form of aqueous gel.

Furthermore, pharmaceutical preparations for dental diseases comprising benzoate, chlorhexidine gluconate and a local anaesthesia are also described in the U.S. Pat. No. 6,365,131.

However, most of the preparations of the prior art have the disadvantage of having an excessively fluid consistency, hence their effectiveness disappears completely within a few hours from application in the patient's oral cavity given that the patient swallows it inevitably.

Thus, in order to overcome these and other disadvantages of the prior art, the present inventors provided an innovative preparation in appropriate association for the topical administration in post-extraction in post-intervention sites, in radicular cleaning, in scaling and root planing and in oral hygiene in general.

A more particular object is to provide an innovative gel capable of solidifying up to taking up the consistency of a soft rubber, whose elasticity can be modulated by the operator depending on the need.

A further object of the present invention consists in formulating the aforementioned gel so that the latter consists of an association of excipients and active ingredients in an appropriate aqueous solution in stomatology in general.

### BRIEF DESCRIPTION OF THE INVENTION

The present invention attains its main objects consisting in providing an innovative gel which is capable of solidifying up to taking up a more or less rubbery consistency and provided with a well-modulable plasticity depending on the type and concentration of the constituents.

Another substantial characteristic of the inventive preparation, also referred to as product in the text, lies in the fact that said gel is applied in the oral cavity or in the area surrounding a tooth or an implant, in a patient requiring aseptic conditions and protection against bacterial infection and/or penetration of food, for the in situ formation of a protective and adhesive barrier, capable of inventively facilitating the bioavailability of medicinal products, antiseptics and anti-inflammatories contained in the preparation.

Another fundamental characteristic in the embodiment of the present invention regards the fact that said innovative gel, once solidified in situ, confers to the product all qualities, such as mucus adhesiveness, elasticity and barrier effects, bacteriostatic action, protection against bacterial intrusion, anti-inflammatory action and the antiseptic action, which confer it considerable effectiveness that is also superior to any similar product of the state of the art. Said innovative product in form of gel, consists in a mixture, which is constituted by polyvinyl alcohol and/or at least one polyacid or the relative salt and/or at least one antiseptic and/or at least one anti-inflammatory and/or at least one tissue regeneration factor, in aqueous solution.

Another characteristic lies in the fact that the inventive preparation is prepared extemporaneously by the health operator, by mixing the constituents, so that it can be adapted, based on the concentrations and the type of constituents, to the various required treatments.

### DESCRIPTION OF SOME PREFERRED EMBODIMENTS OF THE INVENTION

In a preferred embodiment according to the present invention, polyvinyl alcohol has a molecular weight comprised between 10 and 1,000,000, preferably greater than 10,000 and it has a concentration comprised between 0.0001% and 30% w/w and preferably lesser than 4% w/w.

In another preferred embodiment according to the present invention, polyacids and their salts are selected from among the group consisting of polyacrylic acid and its monovalent salts and they have a molecular weight comprised between 10 and 5,000,000 and with a concentration comprised between 0.0001% and 30% w/w.

In a further embodiment according to the present invention, the polyacids and their salts are selected from among the group consisting of alginic acid and its alginates of monovalent salts and they have a concentration comprised between 0.0001% and 30% w/w.

Another embodiment according to the present invention provides for that said polyacid salt is the high molecular weight sodium alginate, with viscosity greater than 40 centipoises at 20° C. in solution at 1.5% w/w and with a concentration comprised between 0.0001% and 5% w/w.

In a further preferred embodiment according to the present invention, the antiseptic is selected from among the group constituted by chlorhexidine gluconate or di-gluconate, chlorhexidine dihydrochloride, iodine, silver ions and any of their combinations and it has a concentration preferably comprised in the range between 0.0005% and 25% by weight, more preferably between 0.002% and 3% by weight.

In another preferred embodiment according to the present invention, the anti-inflammatory is selected from among the group constituted by acetylsalicylic acid and flurbiprofen and any of their combinations and it has a concentration preferably comprised in the range between 0.001% and 30% by weight, more preferably between 0.5% and 10% by weight.

In another preferred embodiment according to the present invention, the tissue regeneration factor hyaluronic acid and it has a concentration preferably comprised in the range between 0.001% and 30% by weight, more preferably between 0.001% and 2% by weight.

According to a particularly preferred embodiment according to the present invention, the ideal preparation is a mixture in polyvinyl alcohol aqueous solution with a molecular weight of about 90,000 at the concentration of about 2% by weight, of polyacrylic acid with a molecular weight of 400,000 at a concentration of 0.5% p/p, of sodium alginate at the concentration of about 1.5% by weight, of chlorhexidine gluconate at the concentration of about 2% by weight and of flurbiprofen at the concentration of about 5% by weight.

As previously specified, though the preparation is preferably obtained extemporaneously by the health operator, suitably modulating the type and the concentration of the constituents, the preparation can also be advantageously obtained in form of a bi-component, i.e. a gel and cross-linking solution in which case several preservatives such as parabens of the propyl paraoxybenzoate and/or methyl paraoxybenzoate type will be required with the aim of obtaining a correct pharmaceutical procedure.

As previously mentioned, the preparation which is in form of an inventive aqueous gel, is deposited in the interdental spaces of the patient suffering from inflammation, gingival recession, periodontal pockets or peri-implant fistulas, so as to fill them and adapt to their shape; in addition, this can be deposited in proximity of the gingival collar and left to cross-link spontaneously, so as to take up the rubbery state.

It should be observed that the inventive preparation attains its main objects even considering the fact that it is advantageously capable of performing three actions. First and foremost, said inventive preparation serves as a protective and bacteriostatic barrier, which combined, secondly, to a bio-adhesive action, in the application area, prevents the infiltration of food residues and bacteria which often are source of infection and/or inflammation. In addition, there is the bactericidal action of the antiseptic, which is released over time by the rubbery gel thus suppressing the microbial load present in the site of administration and around it. The presence of polyvinyl alcohol confers to the film, or to the obtained agglomerate, an elasticity or rubbery solidity (with strong resistance to extension and stress) and adhesive properties which allow the action of sealing the sites of intervention as well as the protection thereof against infiltrations of food and bacteria. In addition, the bacteriostatic properties of polyvinyl alcohol and polyacrylic acid prevent the diffusion of bacteria towards the site of administration. The anti-inflammatory properties of polyvinyl alcohol contribute towards the restoration of the natural physiological conditions with regrowth of the tissues attacked by unfavourable hygiene conditions.

In addition, the product according to the present invention performs an action of sealing and protecting against infiltration of food and bacterial load in any oral site, such as for example in case of implants for dental prosthesis, after-surgery healing of tissues, fistulas and gingival recession.

The product according to the present invention is also recommended for dental and oral hygiene with need for prevention and protection against infiltration of food and bacterial load between one tooth and the other and/or between the gums and the tooth like in radicular cleaning as well as scaling and root planing.

Advantageously, the product according to the present invention allows both the antiseptic to be highly effective at suppressing the bacterial load and the anti-inflammatory to act in the administration site, due to their high local concentration maintained constant over time for several days. To this end, clinical studies have revealed that the effectiveness of the product according to the present invention is even superior to the use of the antiseptic and/or pure anti-inflammatory (100%).

Following studies and tests, polyvinyl alcohol by means of its long chain, in the high molecular weight forms, acts as a bond between the molecules of polyacid or its salt. Thus, the solidifying and elasticising function, which confers a high mechanical resistance and elastic softness to the gel, with an elongation at fracture of more than 100%, derives from its high molecular weight polymeric long chain form, preferably not lesser than 10,000. In such case, polyvinyl alcohol preferably should not exceed the concentration of 4%, in that this would cause the spontaneous solidification of the gel, thus preventing the commercial use of the product which requires stability over time.

The specificity of use of high molecular weight polyvinyl alcohol, lies in the fact that it is used to confer to the invention bio-adhesion to the dental tissues and thus confer to the gel properties for facilitating the transfer of the antiseptic and the anti-inflammatory towards the tissues. Furthermore, bio-adhesion confers to the gel the capacity to seal the administration site protecting it against the entry of bacteria and food fragments.

Though up to now we have considered the use of said inventive preparation for prophylactic/therapeutic purposes, said invention can also be advantageously used for the cosmetic treatment, in which case said product will also contain whiteners for the dental surfaces based on peroxides, hydrogen peroxide and carbamide peroxide.

### EXAMPLES

The examples below, which regard the preparation, characterisation and use of the product according to the invention, are provided solely for illustrative purposes and shall not be deemed to limit the scope of protection according to the present invention, as defined in the attached claims, in any manner whatsoever.

### Example 1: Preparation

The gel was obtained by dissolving polyvinyl alcohol, with molecular weight equivalent to 90.000, at a concentration of 2% by weight, in water. The solution was stirred up to obtaining a homogeneous solution, followed by the addition of polyacrylic acid with molecular weight of 400,000 at the concentration of 0.5% w/w and sodium alginate at a concentration of 1.5% by weight. Chlorhexidine gluconate at a concentration of 2% by weight and, lastly, acetylsalicylic acid at the concentration of 10% w/w, was added to the entirety.

### Example 2: Administration

The gel containing chlorhexidine gluconate, prepared according to Example 1, was introduced into the interdental spaces (or administered around dental implants) by means of a syringe with bevelled needle. Once solidified in loco, the gel prevented food residues from penetrating between the teeth or in the vicinity of the implant. Such treatment is recommendable after performing the radicular cleaning, polishing the roots, curettage, disinfection of periodontal pockets and/or scaling and root planing top avoid frequent recurrence of the disease.

Administration can be expanded by administering a thin layer of gel at the level of the gingival collar (between the gums and teeth) using a small brush, forming a sealing film and antiseptic sealing, for the restoration and maintaining the health conditions of the gums and the root of the tooth.

### Example 3: Characterisation of the Plastic Film

The rubbery film obtained through the procedure described in Example 2 is characterised by elasticity and capacity to release the chlorhexidine gluconate contained therein. Measurements of the elastic elongation at fracture in gel samples were carried out, resulting greater than 100%. The release of chlorhexidine gluconate revealed a duration exceeding 7 days.

### Example 4: Preliminary Clinical Studies

Preliminary clinical studies, carried out with the product of Example 1, revealed the maintenance of a longer optimal oral health after scaling and root planing sessions, radicular cleaning, polishing the roots, curettage, disinfection of the periodontal pockets, thus eliminating the need for repeating the treatment within a short time. The product also revealed to be useful for curing or preventing fistulas, cysts or dental infection after extracting a tooth or a major dentistry intervention, infections in carriers of fixed or moveable dental prosthesis.

### Example 5: Alternative Preparation

The gel was obtained by dissolving polyvinyl alcohol, with molecular weight equivalent to 90.000, at a concentration of 2% by weight, in water. The solution was stirred up to obtaining a homogeneous solution, followed by the addition of hyaluronic acid at the concentration of 0.2% w/w, flurbiprofen at the concentration of 5% w/w and sodium alginate, with specific viscosity of 4,000 cP, at a concentration of 2% by weight. Chlorhexidine gluconate at a concentration of 2% by weight and, land chlorhexidine dichlorhydrate at a concentration of 3% by weight, was added to the entirety.

The administration of the product, according to Example 2, proved to prevent gingival recession and formation of periodontal pockets facilitating the resolution thereof after surgical treatment.

Though the present invention has been described by way of non-limiting example, according to some preferred embodiments, it should be observed that any variations and/or modifications may be carried out by a man skilled in the art without departing from the relative scope of protection, as defined by the following dependent claims.

1. A product for hygiene, cosmetics, or oral or dental care comprising a mixture, in a polyvinyl alcohol aqueous solution, of one or more polyacids or their salts and one or more antiseptics and/or anti-inflammatories, wherein the antiseptic is selected from among the group consisting of chlorhexidine gluconate, chlorhexidine dihydrochloride, iodine, silver ions and any combination thereof.

2. The product according to claim 1, wherein the polyvinyl alcohol of the polyvinyl alcohol aqueous solution has a molecular weight comprised between 10 and 1,000,000, and a concentration comprised between 0.0001% and 30% w/w.

3. The product according to claim 13, wherein the one or more polyacids or their salts is selected from among the group consisting of polyacrylic acid and its monovalent salts and has a molecular weight comprised between 10 and 5,000,000 and a concentration comprised between 0.0001% and 30% w/w.

4. The product according to claim 1, wherein the one or more polyacids or their salts is selected from among the group consisting of alginic acid and its alginates of monovalent salts and has a concentration comprised between 0.0001% and 30% w/w.

5. The product according to claim 1, wherein the one or more polyacids or their salts is a polyacid salt in the form of a high molecular weight sodium alginate, with viscosity greater than 40 centipoises at 20° C. in solution at 1.5% w/w and with a concentration comprised between 0.0001% and 5% w/w.

6. The product according to claim 13, wherein the one or more antiseptics and/or anti-inflammatories is an antiseptic selected from among the group consisting of chlorhexidine gluconate, chlorhexidine dihydrochloride, iodine, silver ions and any combination thereof.

7. The product according to claim 1, wherein the one or more antiseptics and/or anti-inflammatories is an anti-inflammatory consisting of acetylsalicylic acid or a combination of acetylsalicylic acid and flurbiprofen.

8. The product according to claim 7, wherein said product is a gel which, from a fluid-viscous state, becomes a rubbery and elastic solid resistant against the perforation, penetration of food fragments and extension.

9. A method for in-situ formation of a protective and adhesive barrier in a patient requiring aseptic conditions and protection against bacterial infections or penetration of food fragments, comprising providing the product of claim 8, and applying said gel into the oral cavity or into an area surrounding the tooth or an implant of the patient.

10. The product according to claim 8, wherein the gel contains a factor stimulating tissue regeneration.

11. The product according to claim 10, wherein the factor stimulating tissue regeneration is hyaluronic acid.

12. The product according to claim 8, wherein the gel is used for cosmetic treatment.

13. The product according to claim 12, wherein the gel used for cosmetic treatment comprises whiteners for dental surfaces based on peroxides, oxygenated water and carbamide peroxide and any combination thereof.

14. The product according to claim 13, wherein the gel contains preservatives for the stability of the product over time.

15. The product according to claim 14, wherein the preservatives belong to the class of parabens, same case applying to propyl paraoxybenzoate and methyl paraoxybenzoate and any combination thereof.

16. The product according to claim 1, wherein said product is obtained extemporaneously by a health operator, by mixing all or part of the aforementioned constituents.

17. The product according to claim 1, wherein said product is obtained by using the one or more polyacids or their salts and the one or more antiseptics and/or anti-inflammatories are in form of a bi-component comprising a gel and cross-linking solution.

18. The product according to claim 1, wherein polyvinyl alcohol has a molecular weight greater than 10,000 and a concentration less than 4% w/w.

19. The product according to claim 13, wherein the one or more polyacids or their salts is selected from among the group consisting of polyacrylic acid and its monovalent salts and has a molecular weight comprised between 10 and 5,000,000 and a concentration comprised between 0.0001% and 30% w/w.

20. The product according to claim 2, wherein the one or more polyacids or their salts is selected from among the group consisting of alginic acid and its alginates of monovalent salts and has a concentration comprised between 0.0001% and 30% w/w.
